(19) United States
(12) Patent Application Publication
(10) Pub. No.: US 2024/0212826 A1
(43) Pub. Date: Jun. 27, 2024

(54) ARTIFICIAL CONVERSATION EXPERIENCE

(71) Applicant: XOLTAR INC., West Hartford, CT (US)

(72) Inventors: Asaf SHILONI, Tel Aviv (IL); Zachy HERSHKOVICH, Zichron Yaakov (IL); Tal MELLER, Tel Aviv (IL)

(73) Assignee: XOLTAR INC., West Hartford, CT (US)

(21) Appl. No.: 18/594,685

(22) Filed: Mar. 4, 2024

Related U.S. Application Data
(63) Continuation of application No. PCT/IL22/50968, filed on Sep. 5, 2022.
(60) Provisional application No. 63/240,918, filed on Sep. 5, 2021.

(51) Int. Cl. G16H 20/70; A61M 21/00; G06V 40/16; G10L 15/06; G10L 15/18; G10L 15/22; G10L 25/63

(52) U.S. Cl. CPC G16H 20/70; A61M 21/00; G06V 40/175; G10L 15/063; G10L 15/1815; G10L 15/22; G10L 25/63; A61M 2021/0027; A61M 2021/005

(57) ABSTRACT

The invention consists of an artificial conversation agent adapted to conduct conversations using natural language in order to exchange information and otherwise interact with human users, generally including an avatar. This agent is intended to be useful for tasks requiring human interaction such as behavioral modification therapies, continuous monitoring or personal assistance. Many operations and interactions that are usually performed with or using a therapist, customer service representative or other human agent, may be performed using the agent of the invention. For instance, the system is useful for helping subjects maintain fitness or training regimens, quitting smoking or other addictions, sticking to medication programs, changing eating or other habits, overcoming phobias, booking tickets and reservations, providing and receiving information in such contexts as hotel lobbies or airports, and in general providing the ability to interact intelligently with or as human beings in a variety of contexts and their combinations.

### CROSS-REFERENCE TO RELATED APPLICATIONS

This application is a continuation of International Application No. PCT/IL2022/050968 filed Sep. 5, 2022, and claims priority of U.S. Provisional Application No. 63/240,918 filed Sep. 5, 2021, the contents of which are hereby incorporated by reference.

### FIELD OF THE INVENTION

The present invention relates generally to the field of intelligent agents, and in particular to artificial conversational agents.

### BACKGROUND OF THE INVENTION

A number of systems exist for operating intelligent agents capable of generating artificial conversation, ranging from text chatbots giving stock replies to a fixed set of questions, to artificial personal assistants that can be controlled with voice commands and can carry out tasks over the net such as ordering tickets or searching for specific pieces of information, to artificial agents having specific goals to carry out and the ability to sense their environments and adapt to them in an attempt to achieve these goals.

Many of these systems are adapted to take input from a single modality such as text or audio. Audio is generally converted to text by voice-to-text means, and the text (whether input directly or by way of voice-to-text) is parsed, and a text reply or replies is generated. This text may then be converted to audio using various text-to-speech means.

Common architectures used for intelligent agents include:
Reactive agents, which use a direct mapping from situation to action;
Belief-desire-intention agents, which make decisions based on data representing the beliefs, desires, and intentions of the agent; and,
Layered architectures, which make decisions via a series of layers which reason about the environment at different levels of abstraction.

An agent generally is equipped with a set of sensors for perceiving the environment, and actuators for acting upon it. Intelligent agents generally possess an internal model of the environment that encapsulates all the agent's beliefs about the world, and the goals are modeled in terms of an objective function.

Russell & Norvig (Artificial Intelligence: A Modern Approach, 2021, “http://aima.cs.berkeley.edu/”) introduced the following five classes of agents, based on their degree of perceived intelligence and capability:
1. Simple reflex agents act only on currently perceived data, and can be implemented in terms of ‘if-then’ behavior.
2. Model-based reflex agents store a model of the world within the agent which includes things that cannot directly be perceived, but other than this acts like a simple reflex agent insofar as it acts in an ‘if-then’ paradigm.
3. Goal-based agents evaluate situations in terms of desirability. This provides the agent a way to choose among multiple possibilities, selecting the one which reaches a goal state. Search and planning are subfields of artificial intelligence devoted to finding action sequences that achieve the agent's goals.
4. Utility-based agents allow a continuum of values instead of the ‘binary’ goals of goal-based agents, which are either achieved or not.
5. Learning agents can operate in unfamiliar environments and become more competent than initial knowledge alone would allow. A learning element uses some estimate of how the agent is performing and determines how the agents' actions should be modified to better increase utility or satisfy goals.

Intelligent Personal Assistants (IPAs) are familiar in smart phones (Apple's Siri, Google's Assistant, Microsoft's Cortana, Samsung's Bixby) and other devices (Amazon's Alexa, Google's Home). These devices generally are competent at voice recognition and carrying out various internet-based tasks, like online shopping, playing music, modifying schedules, sending messages, offering answers to simple questions, and controlling household appliances. Generally, these all involve text-based interactions (after speech has been converted to text) with entities designed to carry out tasks, with no intent or agenda of their own.

A number of patents have been filed dealing with innovations in this space. Most deal with particular subproblems involved, such as voice detection and noise reduction, speech-to-text conversion, natural language understanding, and the like. A few are more general, dealing with issues of overall architecture or operation of an intelligent agent. For instance, US20120016678A1 (Apple, Intelligent Automated Assistant) “engages with the user in an integrated, conversational manner using natural language dialog, and invokes external services when appropriate to obtain information or perform various actions . . . using any of a number of different platforms, such as the web, email, smartphone, and the like, or any combination thereof. In one embodiment, the system is based on sets of interrelated domains and tasks, and employs additional functionally powered by external services with which the system can interact.”

This application claims
“. . . an input device, for receiving user input;
a language interpreter component, for interpreting the received user input to derive a representation of user intent;
a dialog flow processor component, for identifying at least one domain, at least one task, and at least one parameter for the task, based at least in part on the derived representation of user intent;
a services orchestration component, for calling at least one service for performing the identified task;
an output processor component, for rendering output based on data received from the at least one called service, and further based at least in part on a current output mode; and
an output device, for outputting the rendered output.”

While doubtless a sophisticated piece of technology, this actually is at the lower end of the ‘intelligent agent scale’ listed above, being an implementation of a either a simple reflex-based agent or a model-based reflex agent.

As another example, WO2018/093770 (IPSoft, Generating Communicative Behaviors for Anthropomorphic Virtual Agents Based on User's Affect) provides for “automatically generating . . . facial expressions, body gestures, vocal expressions, or verbal expressions for a virtual agent based on emotion, mood and/or personality of a user . . . systems and method for determining a user's emotion, mood and/or personality are also provided.” This approach of mirroring a user's perceived emotions is found in many patents and while a useful approach to build trust and facilitate communication, it is only one approach and others may be more effective in certain situations, as will be further detailed below.

As an example of prior art dealing more generally with problems associated with operating an intelligent agent consider US 2017/0206095 (Samsung, Virtual Agent) which provides a method for operating a virtual agent based on determining an ‘interaction context’, based on which the agent state is determined and behaviors are selected, in this case behaviors involving change in appearance of the virtual agent or generation of audio. The interaction context is apparently obtained through video, audio, or other sensor information about the user, other people, or the user's surroundings, and involves interpretation of elements of the environment such as lighting, type of room, and whether the user is alone or with others. Responses are determined in part based on these factors. Minor stimuli observed can be responded to, e.g. an expression of surprise after seeing a user drop a glass.

Virtual agents with an agenda or ‘drive’ are also found in the prior art, for example US 2019/0042988 (Telepathy Labs, Inc. Omnichannel, Intelligent, Proactive Virtual Agent) which describes a multi-channel, proactive virtual agent wherein a user has a conversation with the agent to interact with structured and unstructured data of an enterprise. The agent proactively reaches out to employees about information or developments that are relevant to a business.

While such systems have advanced greatly in recent years due to corresponding advances in NLP and machine learning, a number of shortcomings remain that limit their efficacy in many real-world situations.

Amongst these shortcomings are the ability to deal effectively with information coming from several sources and priors, for instance audio, text, video, location, history, and the like, and to transition between these, as necessary. Further there appears no provision to deal in modular fashion with different contexts such as location or use case (e.g., ticket agent vs. hotel concierge vs. salesperson). Finally, there is in general no provision for use of techniques developed in behavioral psychology and related disciplines in the operation of such agents.

### SUMMARY OF THE INVENTION

The invention consists of an artificial conversation agent, including an avatar useful for such tasks as therapies, including but not limited to cognitive behavioral therapy, other forms of therapy including psychoanalysis, psychodynamic therapy, behavioral therapies, cognitive therapies, humanistic therapy, holistic therapy, integrative therapy, group therapy, other forms of therapy, counseling including event counseling and performance counseling, motivational interviewing; practical solutions, such as booking tickets and reservations, ordering items, paying for goods or services, and providing and receiving information from open sources of from accessible databases; and in general providing the ability to interact intelligently with human beings in a variety of contexts, using a variety of input and output modes in parallel. Input modes can include video from one or more cameras, audio from one or more microphones, user data from any source (be it local or distributed), historical and current context data such as current weather, time of day, location, and predicted future data of this sort such as predicted weather, as will be discussed later in the detailed description.

To deal with this large amount of data the system is roughly modeled on the nervous system and includes a number of subsystems each designed to be operated independently and in parallel. For instance, a video camera will in many cases be used with the system. The video data may be sent through a number of preprocessing steps such as downsampling and white balancing. This processed video may then be analyzed by several subsystems, such as a detection and segmentation subsystem (e.g., adapted to find bounding boxes each containing one or more people) which then sends segmented images to several ‘consumers’ of this data, for example, an emotion detection subsystem, an age detection subsystem, and a gender detection subsystem.

Each subsystem has a number of inputs and outputs, and these outputs can in turn be used by other subsystems as in the previous example. This high degree of modularity is one novelty of the system, and it allows for the system to handle a wide variety of use-cases without additional effort.

Further building upon the nervous system analogy, the responses of the system are divided into ‘instinctual’ or rapid, spontaneous responses to events, and ‘reasoned’ responses requiring analysis and decision making based on various data, these roughly corresponding to the simple- and model-based reflex agents mentioned above. However, there is also provision for integrated goal- and utility-based operation, as will be described later.

As an example of an instinctual response, if a user of the system is observed to sneeze (this observation for example being made by video, audio, motion, or a combination of the two), the agent may respond appropriately with an audio output, for example with the statement ‘Gesundheit’ if the location of the interaction is a German-speaking region. This statement may be made in text or audio as appropriate to the situation. It is an immediate, almost history-blind but location-aware response to a certain input.

On the other hand, if the user for instance asks a question, the response thereto involves a number of factors not required by ‘instinctual’ responses as described above, including context awareness, memory, and other information. If the input is in the form of speech, this is generally converted to text, which is then input into further subsystems as described above, which are adapted to generate appropriate responses. Take for example an agent of the invention acting as a medical adherence agent. If a user asks the agent ‘Where can I get a refill for my medicine?’, the speech is converted to text using any means known in the art. Then NLP of any variety known in the art reaches the conclusion that the user is looking for a pharmacy in his local environment, and user data may then be consulted to determine the user's preferences (e.g. brands of pharmacies, favorite locations and opening hours, and the like). If such user data is not available, then it is within provision of the invention that they be determined, for instance by a process of questioning or probing of the user by the agent. A number of options may thereby be determined, and presented to the user for instance by means of a list, points on a map, a set of images, or the like. The user may then choose one of these options, which is then booked, called, or otherwise contacted by the agent if appropriate, and the user agent may further offer help getting a delivery, or getting to the pharmacy, for example offering to call a taxi, Uber, or other transport, providing walking or public transport directions, or the like.

This is an example of a prolonged interaction with history, where one question on the user's part leads to a response on the agent's part, this response includes a question for the user, and so on. It is a feature of the invention that this ‘local context’ of the conversation be stored in the form of short-term memory, and used to inform the future responses of the agent.

The ability to use two or more different behavioral modes as necessary, in a modular fashion, is a key aspect of the invention. When dealing with a user for the first time, there is less context available. As the interaction progresses, the system is able accumulate information concerning user intent, context, user behavior and so on in order to build reasoned responses. This accumulated information may be stored for use the next time this particular user interacts with the system, or may be stored for use in aggregate form (for example wherein likely user needs, behaviors, or responses may be stored in the form of frequency, histogram or average data).

Goal/utility-based behavior is made possible in the system by use of an ‘adaptive agenda’.

This is a set of goals or utility functions operating on states which can control the overall arc of the agent's behavior. An agent with the goal of (for instance) selling a car on a certain plan may conduct a lengthy conversation with a potential client, answering questions and giving details about various options, all with the ultimate goal of selling the plan in mind. Various ‘detours’ along this route (e.g. answering related or unrelated questions, responding appropriately to pauses or interruptions, making small talk, etc.) will not derail the system since the ultimate goal remains in force, until changed. Changes in the agenda can occur when new information becomes available. For instance, if the potential client reveals that he/she is from overseas then the goal may be changed to selling a rental car plan instead of selling a car outright. This new goal then takes effect and the avatar's actions are modified in keeping with the new goal.

It is within provision of the invention to seamlessly transition between different sensors and communication platforms as the need arises.

It is within provision of the invention to initiate action (for instance a sales solicitation, task initiation, or the like) based on any possible input. For instance, if an agent of the invention that is adapted for medical diagnosis determines that a user has likely suffered a stroke, the system may immediately call for medical assistance, without any further action from the user. As another example, if an agent of the invention that is adapted for the role of a private tutor detects that the user hasn't understood a key concept even after several attempts, the agent may suggest taking a break e.g. to play a video game, before returning to the missed concept at a later stage.

Moreover, it is within the provision of the invention to combine multiple goals into one or several conversations across time, to prioritize, adapt and tailor online, during a session or between sessions, manually or automatically such combinations.

It is within provision of the invention to detect and respond appropriately to ‘unexpected’ changes of context, such as when strangers edge into a conversation between agent and user, the loss of the user's attention, change in focus of the user's conversation, etc.

It is within provision of the invention to improve the performance of existing chatbots and other agents. This can be done relatively easily, given the level of abstraction used by the system. For example, consider an existing agent having a set of relevant business information used by a textual chatbot, e.g. in the form of a script. This script can be copied and integrated into the conversation manager of the invention, and used directly.

It is within provision of the invention to respond to events in the scene, including verbal input from a user but also including any other input detected by the system. Changes in the scene can be integrated into the responses of the system. For instance, if an agent of the system is configured to supervise a student learning a vocabulary lesson requiring the student to write the translation of an English word into French, the agent may move on to the next word if the camera detects that the student has written the correct answer.

It is within provision of the invention to initiate interactions with users, for example by starting conversations with a person or people in the vicinity, writing text to a chat box, calling cellphones or landlines, sending emails, and so on. During these interactions it is further within provision of the invention to follow a proactive agenda, in order to steer the customer to a particular business outcome, like a sale.

It is within provision of the invention to use a variety of inputs including audio, video, chat, and combinations of these.

It is within provision of the invention to adapt to different languages by means of modular system components. For example, the language being used can be represented by various methods including use of a localized dictionary or language-specific NLP module.

It is likewise within provision of the invention to adapt to different businesses and sales models using similarly modular system components, including modules representing business information.

It is within provision of the invention to perform or assist in the performance of various forms of behavior modification, including cognitive behavioral therapy, motivational interviewing, mentoring and coaching procedures and any other treatments intended to improve adherence, compliance, and other aspects of behavior of a subject.

Motivational interviewing, in particular, sits midway between following (good listening) and directing (giving information and advice) and is a particularly apt use of the avatar of the invention, as this therapy is designed to empower clients to change by drawing on their own capacity for change.

In accordance with the aforementioned, it is within provision of the invention to assist in adhering to fitness regimens, diets and other forms of dietary behaviors, weight loss programs, smoking reduction or elimination plans, phobia elimination, substance abuse treatment, addiction treatment, behavioral support, and any other behavior modification technique traditionally involving person-person or person-to-group interaction. This assistance may be single-goal or multiple-goal based, and within this provision is the orchestrating of multiple goals or different therapeutic disciplines or paradigms to a coherent long-lasting relationship, Including the combination of different paradigms between or within conversations.

To facilitate behavior modification of various sorts, it is within provision of the invention to facilitate various forms of emotional connection between avatar and user-companionship, friendship, emotional investment, and the like will generally make behavioral modification techniques used by the avatar more effective.

The foregoing embodiments of the invention have been described and illustrated in conjunction with systems and methods thereof, which are meant to be merely illustrative, and not limiting. Furthermore, just as every particular reference may embody particular methods/systems, yet not require such, ultimately such teaching is meant for all expressions notwithstanding the use of particular embodiments.

### BRIEF DESCRIPTION OF THE DRAWINGS

Embodiments and features of the present invention are described herein in conjunction with the following drawings:

FIG. 1 shows a division into perspective and expressive categories;

FIG. 2 shows a detailed conceptual design of the system;

FIG. 3 shows a block diagram of system components;

FIG. 4 shows a block diagram showing paths for both instinctual and reasoned responses.

### DEFINITIONS

In the following, the term ‘user’, ‘subject’ and ‘patient’ may be used interchangeably to indicate one or more users of the inventive systems and methods.

The terms ‘avatar’ or ‘agent’ refer interchangeably to an animated character having a set of distinguishing characteristics including physical appearance, voice, vocabulary, and modes of response. Such an avatar will be presented using computing means, generally employing duplex audio and video.

### DETAILED DESCRIPTION OF PREFERRED EMBODIMENTS

The present invention will be understood from the following detailed description of preferred embodiments, which are meant to be descriptive and not limiting. For the sake of brevity, some well-known features, methods, systems, procedures, components, circuits, and so on, are not described in detail.

The invention consists of an artificial conversation agent adapted to conduct conversations using natural language in order to exchange information and otherwise interact with human users, generally including an avatar (a visual representation of the agent, for instance an animated face or full 3D model of a human being or other figure). This agent is intended to be useful for tasks requiring human interaction such as behavioral modification therapies and accountability partnership. Many operations and interactions that are usually performed using a customer service representative or other human agents, may be performed using the agent of the invention. For instance, the system is useful for helping subjects maintain fitness or training regimens, quitting smoking or other addictions, sticking to medication programs, changing eating or other habits, overcoming phobias, booking tickets and reservations, providing and receiving information from open or private databases, ordering services or goods, and in general providing the ability to interact intelligently with human beings (and as human beings) in a variety of contexts.

A key provision of the invention is to allow for long-term goal-oriented interactions between avatar and user, that extend beyond a single session or a single goal. This is in contrast to today's chatbots of various sorts, which generally are ‘single-session resolution-driven’ in that they attempt to minimize time-to-resolution insofar as possible, and attempt to do so in single session. Indeed, such chatbots often do not have access to a given user's chat history, and so the interactions are necessarily ‘from scratch’ every time.

In contrast to such ‘history-free’ operation, the avatar of the invention is intended to have characteristics that encourage the formation of a long-term relationship with the user over multiple sessions or interactions, similar to that of a therapist with his/her patient. The formation of this relationship is beneficial in a number of aspects, for instance facilitating behavior modification wherein the user will attempt to please the avatar, even if at a subconscious level.

The avatar's performance may be judged by a set of metrics, albeit rather different metrics than those in operation for chatbots. For example, instead of attempting to resolve issues as fast as possible, the avatar may be rated by the degree to which the user seeks more interaction, their evaluation of the avatar or the experience involving them, or their long-term commitment to the program and the behavioral changes it calls for. It is within provision of the invention that characteristics of the avatar be changed in an attempt to improve such metrics, either in real time or otherwise. Such changes may be implemented on an avatar-by-avatar or user-by-user basis, or may be implemented by use of aggregate statistics combining measurements from multiple avatars and/or multiple users.

It is within the provision of the invention that the avatar be used to track the progress of a user over time in terms of various states or goals. For instance, given a user attempting to stop or reduce smoking, the avatar will beneficially track the amount to which a user continues to smoke, either by interrogation of the user, by reporting by means of other entities (for example, a human partner of the user), or by other detection means. For instance, a sensitive smoke detector with net connection may be used in this case to allow the avatar to more comprehensively monitor a user's smoking habits, subject of course to various privacy laws and permissions that may come into play. Other means for detecting a user's adherence, compliance, or other behavior may also be used, including the use of a smartphone's microphone, camera, inertial measurement unit, GPS, internet interactions, and any other detection means.

Thus, for example, a subject using the system to reduce alcohol consumption may be adapted to monitor the subject's location in an attempt to determine whether he or she is frequenting bars or liquor stores, and for the same purpose, the system may be adapted to monitor the subject's purchases, for instance by means of inspecting the subject's credit card transactions. Access to these data may in many cases be facilitated by means of the smartphone, which is generally provided with GPS capabilities as well as NFC payment means. Other avenues for obtaining such information may also be used in the case that any particular avenue is not available through cellphone means.

While we may refer to smartphone or avatar operation, it should be kept in mind that the system of the invention may be implemented either on cloud servers, on the smartphone itself, or in a hybrid, with any particular action originating from either or both of these sources. Likewise, when we refer to a smartphone, it should be understood that any suitable computing means may be substituted, for instance a tablet, desktop computer, or the like.

It is within provision of the invention that multiple modes of communication be used by the avatar, including but not limited to text or SMS notifications, chats (e.g. WhatsApp™, Telegram™, Slack™), emails, Zoom™ sessions, other online video sessions, voice calls, speech and/or video using a bespoke smartphone app, and so on; this may for example allow a given user to find the most comfortable communications means.

The inventive method is highly modular by design, for instance being adapted to use a variety of input modes in parallel, without being entirely dependent on any of them. A modular set of sensors each with its own interpreter provide input to a contextual center, which also has access to a set of behavioral knowledge modules through a set of abstractions, a set of business knowledge modules also accessed through a set of abstractions, short and long term memories, and a set of media outputs each having its own interpreter adapted to take higher level information and convert it into low-level output. Modes such as video from one or more cameras, audio from one or more microphones, user data from any source (be it local or distributed), and historical and current context data such as current weather, time of day, location, and predicted future data of this sort such as predicted weather may all be used by the system.

In order to make the system as modular as possible it includes a number of subsystems each designed to operate independently and in parallel. Each subsystem has a number of inputs and outputs, and these outputs can in turn be used by other subsystems. Input means may include keyboard and mouse, camera, microphone, position sensors, and any other input means of a human-machine interface; online sources such as APIs, web feeds, databases, and other online sources; as well as radio, telephone, television, and other information channels. Output means may include speakers, headphones, video screens, annunciators, heads-up displays, and any other output means of a human-machine interface, radio and other information channels, and online outputs such as APIs, web feeds, databases, and the like.

The use of modular subsystems has several advantages. First of all, it makes the system resilient, as it is designed to avoid overdependence on any particular input; thus, for instance, if the camera(s) are blocked, the system will continue to operate using the microphone input, albeit with some reduction in response, while if the microphone input is unusable (due to excessive noise or mechanical failure, for instance) the system will fall back to other means, e.g. using the video input for determining speech by means of lip reading, using touch screen input, or by using other inputs.

A second advantage of the overall modular design of the system (beyond the modular nature of the inputs and outputs) is that it is easily adapted for use in a number of different contexts; it can be implemented for instance over the phone (giving customer support, doing sales operations, gathering information, conducting polls, and the like), in a smartphone (e.g., after the fashion of a personal assistant), on the web (e.g. implementing a chat-bot or on-site customer service representative), in a stand-alone kiosk (giving travel information in an airport or train station), taking reservations and orders in a restaurant, acting as a concierge in a hotel (e.g., booking rooms, dealing with reservations and cancellations, etc.) and so on. Any role requiring voice, text, or possibly other modes of interaction, with human beings or possibly with other intelligent agents, is within the purview of the invention.

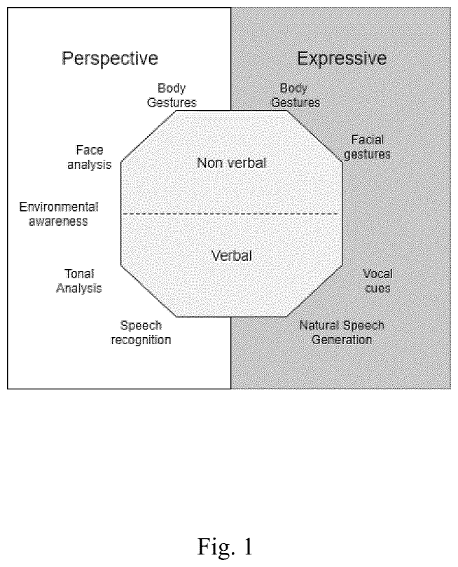

At a high level, the system may be divided into perspective and expressive parts as shown in FIG. 1. The perspective part of the system comprises an input for instance from video and audio input means (for example, camera and microphone) and includes provision for interpreting such input to derive inferences from this data. This may include, for example, interpretation of body language to determine gestures, face analysis to determine mood or emotion, speech recognition for conversion of audio to text, means for determining which of a number of potential users are speaking, and the like. The system also includes provision for various forms of environmental awareness and indirect communication. These analyses may be divided into verbal and nonverbal elements, for example speech recognition being verbal and body language being nonverbal.

The expressive part of the system includes forms of output that the system may generate using video, audio, and other modalities as may be available. For example, the avatar of the system may express intent using body language (e.g. with a nod, shrug, and various facial gestures), other forms of indirect communication, and speech generation; these expressive modalities also may be divided into verbal elements such as speech, and nonverbal elements such as facial gestures.

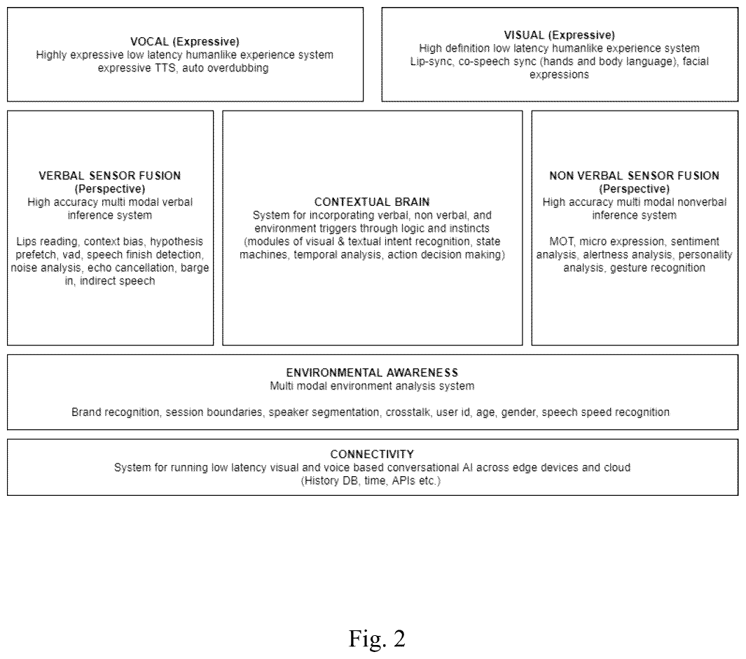

FIG. 2 shows a detailed diagram of the system components. As mentioned, the system incorporates a set of expressive capabilities including vocal output, for example in the form of a human voice speaking in a given language or set of languages.

Verbal and nonverbal input is dealt with using a multimodal inference system incorporating data from all the inputs of the system. Generally, these inputs will include video (where verbal information may still be inferred for example using lip reading), and from audio (such as speech recognition means including voice activity detection (VAD), speech finish/end of statement detection, noise analysis, echo cancellation, sentiment analysis both from content and from tone of voice, and indirect speech detection).

Nonverbal sensor fusion means include multi-object tracking, micro-expression recognition, sentiment analysis, alertness analysis, personality analysis, gesture recognition, and in general any other means adapted to make inferences based on nonverbal cues based on the video, audio, and other input means of the invention.

The system also includes a multimodal environmental awareness module. This module includes provision for detection of such phenomena as session boundaries, speaker segmentation, side speech recognition, crosstalk recognition/filtering, user identification, age determination, gender recognition, co-worker identification, analysis of the speed of a user's speech, brand recognition, and the like. Generally speaking, any element of the environment that can be useful as input for the rest of the system is within provision of the system for detection and use. As mentioned above, the various components of this module (as for all the modules of the system) are themselves designed to be as modular as possible, such that they may be replaced easily if a better-performing or alternative component is discovered. Thus, for example an age-estimation component currently performing at an overall 90% accuracy and requiring 0.1 s, may be replaced by a newly developed age-estimation component e.g. operating at 95% accuracy and requiring 0.05 s. As will be clear to one skilled in the art, this is most easily done if the inputs and outputs are well-defined, in this case for instance the inputs being video and/or audio streams, and the outputs being a gender classification for each observed user or potential user, updated (for instance) once every few seconds.

A connectivity layer is also provided that allows for low-latency visual and voice-based conversational AI across edge devices (such as smartphones, tablets, and the like) and over the cloud. Connection to various communication platforms is also within provision of this layer, including connection to streaming and/or messaging platforms for purposes of input and/or output to/from the system.

The above components all interact with the central ‘contextual brain’ of the system (except for the case of instinctual reactions which are invoked directly by lower-level data processors). The contextual brain is sent inputs from the aforementioned components, and takes action based upon them. This system incorporates verbal, nonverbal, and environmental inputs and acts upon them using a set of expressive engines to (for example) output audio, animate a video character, send data over the internet, and in general operate any actuator that is connected to the system.

The contextual brain may incorporate state machines, meta-analysis, analysis of intent to act, and so on to inform its responses to its inputs.

The responses of the system are divided into ‘instinctual’ or rapid, simpler responses to spontaneous events, and reasoned responses requiring analysis and decision making based on various data.

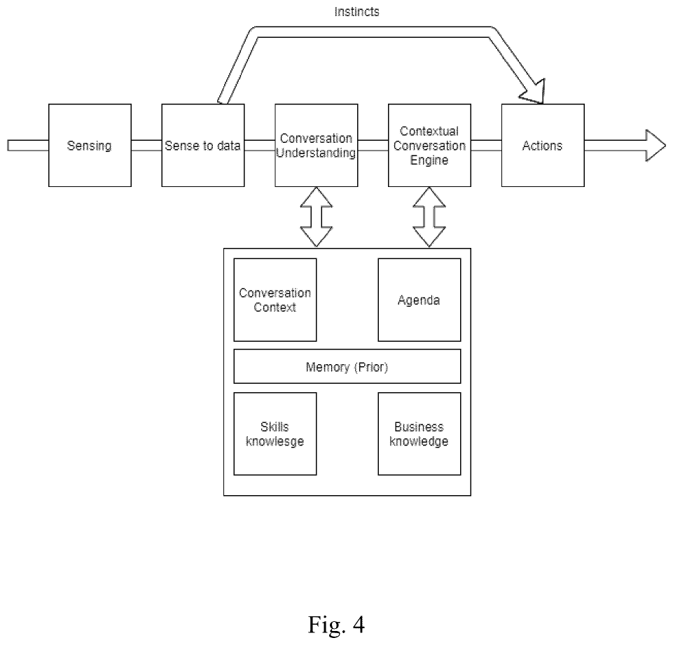

One possible implementation of this system is shown in FIG. 4, where sensing means (microphone, camera, etc.) are converted to data, this being a higher-level representation of what has been sensed (for instance, ‘the user has sneezed’). Data may be acted upon instinctually and converted straight to action, or it may follow a reasoned route, the data first being converted to information, which is then used to inform the context. The ‘contextual brain’ has access to skills or role knowledge (i.e. what actions are appropriate/desirable in a given context) and business knowledge, and updates an adaptive agenda informed by these. The contextual brain uses the various inputs, context, and other memory aspects, to make decisions about most probable states and most desirable goals. These decisions are subsequently translated into actions downstream in the pipeline.

The contextual brain generates text which is then converted to speech by an action engine.

For example, if a user of the system is observed to sneeze (this observation for example being made by video, audio, or a combination of the two), the agent may respond appropriately with an audio output, for example with the statement ‘Gesundheit’ if the location of the interaction or cultural background of the user is German speaking. This statement may be made in text or audio as appropriate to the situation. This response may for instance be stored in the form of a set of ‘stimulus-response’ pairs, where the stimulus in this case could be ‘user sneezed’ and the response could be ‘output an appropriate locale-specific response to a sneeze’, which would take the form of the spoken word ‘Gesundheit’ in a German-speaking region when n audio speaker is available, but would take the form of the printed text ‘Bless you’ in an English-speaking region when no audio system is available but text output is available. The locale-specific responses may be determined for instance using a look-up table, dictionary of key-value pairs, or other means as will be clear to one skilled in the art.

On the other hand, if the user for instance asks a question, the response thereto requires a number of steps not required by ‘instinctual’ responses as described above, including context awareness, memory, and other information. If the input is in the form of speech, this is generally converted to text, which is then input into further subsystems as described above, which are adapted to generate appropriate responses. For example, if the user asks a user agent of the system ‘Where can I get a bite to eat tonight’, the speech is converted to text, and NLP/NLU estimates the user intent—that the user is looking for a restaurant in his local environment.

To continue the example, once user intent has been estimated, user data may be consulted to determine the user's dining preferences (e.g. types of restaurants, price ranges, eating restrictions, and the like). If such user data is not available, then it is within provision of the invention that they be determined, for instance by a process of interrogation of the user by the agent. A number of options may thereby be determined, and presented to the user for instance by means of a list, points on a map, a set of images, or the like. The user may then choose one of these options, which is then booked, called, or otherwise contacted by the agent of the invention if appropriate, and the agent may further offer help getting to the restaurant, for example offering to call a taxi, Uber or other transport, providing walking or public transport directions, or the like. Were this interaction to be interrupted (for instance by another sneeze), an appropriate instinctual response would then be made once again, and the dining interaction resumed.

This is an example of a prolonged interaction with history, where one question on the user's part leads to a response on the agent's part, this response including a question for the user, and so on. It is a feature of the invention that this ‘local context’ of the conversation be stored and used to inform the future responses of the agent by means of a short-term memory, as will be explained below. This example also shows an example of the adaptive agenda, where an overall goal of helping the user achieve his/her goals leads the agent to (in this example) determine dining preferences, then reserve a table, then call a taxi. Once the user intent for dinner has been estimated, the agent adopts this goal into its agenda and attempts to help the user achieve it, even in the face of difficulty, interruption, and other distractions.

Such goal-driven behavior (as opposed to strictly reactive/instinctual/model-based behavior) is useful in a number of different contexts, for instance sales, customer service, instruction, task supervision, and the like. In the case of sales, several goals may be in operation such as to close a sale, provide a brand impression, give information concerning a particular deal, and the like. Customer service may likewise benefit from goal-oriented behavior such as keeping the customer happy, adopting the customer's goals as above and attempting to understand and solve their issue, etc. As will be clear to one skilled in the art, the goals mentioned may involve sub-goals that must be obtained first, in some cases the sub-goals being strictly necessary and in other cases one of several sub-goals being sufficient for progress towards the parent goal.

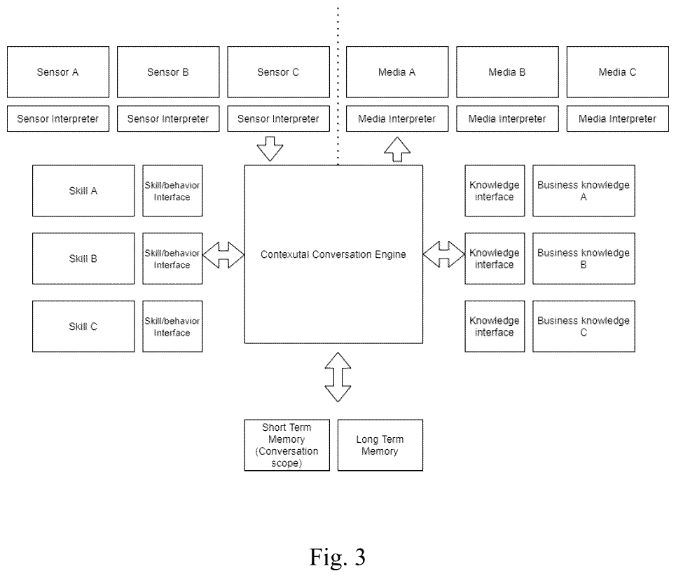

FIG. 3 shows a block diagram of the system which emphasizes the modular nature of the system. A set of sensors A, B, C, etc. are each used as inputs to a set of interpreters. Sensors may comprise for instance cameras, microphones, and other human-machine interface means. The interpreters may be for instance speech-to-text means, gender detectors, age detectors, gesture interpreters, and the like. The outputs of these interpreters serve as inputs to the contextual brain of the system.

Behavioral knowledge and various skills and abilities are represented in the system by another set of blocks, Behavioral Knowledge A, B, C etc. These interact with the rest of the system through abstraction layers, which reduce the behavioral knowledge to higher-level, conceptual messages. For instance there may be a behavioral knowledge module concerning the user's state of mind—relaxed, annoyed, rushed, etc.; in essence, a ‘mood detector’. Inputs to such a module might be the textual representation of the user's speech, as well as intonation, speed of speech, body language and facial gestures, and all of which are input to the system via the microphone and camera sensors, interpreted by the various interpreters, passed to the central contextual center, and from there sent to the ‘annoyance’ behavioral knowledge module, which is tasked with making a determination that the user is (for instance) annoyed. This determination may be output from the ‘mood detector’ in the form of a set of class probabilities (e.g. 0.9 annoyed, 0.8 rushed, 0.01 relaxed) and sent to the contextual center, which can now take this into account by a. putting this information into the short-term memory, and b. by modulating its responses appropriately. This may include, for instance, the use of apologetic phrases, modified speech, or the like.

A further set of modules is used to represent various forms of business knowledge. For instance, information concerning a set of local venues, services and service providers may be included in one module to allow this type of business information to be used by the rest of the system. Take as an example that a user expresses a desire to eat, (for example by means of information input to the cameras/microphones of the system, converted to text by the various sensor interpreters, and from there input to the contextual center). The contextual center can then use information from the ‘venues and services’ business knowledge module to generate a set of relevant dining venues as a response to a query from the contextual center. The query may include context information which may be e.g. pulled from other sources, including other behavioral knowledge modules, and short/long term memory. For example, the current time is available from short term memory or a system clock, and what this particular user likes to eat may be pulled from a behavioral knowledge module about particular user likes and dislikes. If the hour is 7 PM and the user likes Chinese food, these may be included in the query to the ‘venues and services’ business knowledge module, or alternatively only the type of venue (places to eat) may be sent, the entire list of relevant venues is sent back, and this list is filtered by the contextual centers using the time and eating preference information from short and/or long-term memory. If, for example, the user expresses a desire to eat for the second night in a row, then relevant information may be taken into account along the following lines. The audio and video are translated into text as before through the sensor interpreters, and this text is input into the contextual center. The long-term memory retains information about preceding transactions including last night's suggestion of Chinese food, which the system may conclude was acted upon, since the user ordered an Uber to the second Chinese food venue offered. Remembering that the user apparently ate Chinese last night, the behavioral knowledge ‘likes and dislikes’ module is queried for other food preferences, and if for instance it is found that the user also likes fried chicken, the ‘venues and services’ module can be queried to find a venue serving fried chicken for dinner.

When a response has been generated by the contextual center (for instance in the form of a piece of speech to be generated, an image or set of images, map location, etc.), it is output to one or more of the appropriate media (e.g. speaker, screen, or any other human-machine interface output means) by way of a media interpreter. Thus for instance if the text to be output is ‘How about some fried chicken for dinner?’, this is sent to a text-to-speech media interpreter. This module incorporates information from the avatar to generate the appropriate audio waveform and avatar animation. The output will generally also take into account variables such as the user's state of mind (thus speech to an annoyed user may be short and to-the-point while speech to a relaxed user may be slower, in general matching the user's speed of speech). Once speech and animation is output, the high-level representation of the output is stored in the short term memory, and further input can then take this into account. For instance, a reply ‘OK’ to ‘How about some fried chicken for dinner’ may be interpreted as the user agreeing to a fried chicken dinner, since this is a possible appropriate response to the most recent event stored in short term memory. A set of fried chicken venues may then be presented to the user either by means of text, a set of images representing different restaurants in a grid, points on a map, or the like, with auxiliary information such as cost, diner ratings, and the like made available by means as will be familiar to those skilled in the art.

The business modules may also incorporate various goals that will be achieved by the adaptive agenda mechanism implemented inside the contextual brain. For instance, one possible use of the intelligent agent of the invention might be to drive sales on a website. Thus the business module in this case may include not only a list of available goods and/or services and information about these, but also a set of goals to achieve, for instance to interest the user in a given product, to expose the user to certain set of images or information, and the like. The contextual center will take these goals into account and attempt to achieve them within the known context, for instance taking into account the user's past buying behavior in order to determine an ‘approach’ (eg. hard sell vs. soft sell, areas of interest, etc.) Aggregate past behavior of cohorts of users may also be used, for instance the past behavior of users in the same age or other demographic as the current user being served.

The short and long term memories may be represented in terms of sets of processed data, which for instance have been categorized, classified, or otherwise reduced to representative form. The short term memory may be converted into long term memory as appropriate. F or instance at the end of every interaction with a given user, the events associated with that interaction are all taken from short term memory and stored in the long term memory. The events of the long term memory may be utilized to inform one or more other modules; for instance, feedback from multiple users concerning certain eating venues may be fed into the ‘likes and dislikes’ behavioral module, allowing that module to build up a long-term profile of which restaurants people generally prefer, or (for example) which type of people prefer which types of restaurants.

There are modules of ‘business knowledge’ comprising knowledge and context about a given business, and there are modules of ‘role information’ comprising information about the goals, expected behavior, context, etc. for a given role. Business knowledge modules may include price information concerning what products are available, what they look like, where they can be obtained, prices, etc. Role information may include goals such as attempting to sell a given product, attempting to oversee a given process, providing information in a hotel lobby, taken reservations at a restaurant, and so on.

It is within provision of the invention to use the same avatar and voice for each interaction with a given user, to provide a sense of continuity and familiarity to the user. However, the avatar(s) used in a particular interaction are in general ‘swappable’ in the sense that different characters may be used to interact with a given user, these avatars for instance being chosen from a library of possible characters, either chosen by the user, or chosen by the system automatically based on predicted user preferences (e.g. middle-aged female avatar for hotel lobby, older male avatar for financial services, cartoon characters for kids, etc.) For the purposes of the invention, the avatar can include a particular voice matching the video character, as well as a possible set of behaviors including stock phrases, physical gestures, etc. The avatar, including its appearance, voice, and set of behaviors in different situations (a sort of ‘personality’) may thus be easily swappable, with various factors informing the choice avatar, including locale, gender, age, and possibly race of the user, personal preference of the user, etc. The behavioral features (‘personality’ as mentioned, voice, language, etc.) can in large part be decoupled from the rest of the system, and instead are stored as part of the avatar.

The modules of the system may be implemented using deep learning, for example with CNNs and other networks used as classifiers, and/or RNNs adapted to take actions based on the current input and history.

It is within provision of the invention that it be independent of use case, insofar as possible.

Thus, for instance the use case of hotel concierge and ticket agent may both be handled by the intelligent agent of the invention; the differences may amount to as little as choice of avatar used and the business knowledge modules employed. The rest of the system (sensors and their interpreters, contextual center and so on) may all be identical. Even if the particular sensors are different, the remainder of the system (beyond the sensor-specific interpreters/re-processors that may be required for particular sensors) may remain the same and achieve the same performance with a different set of sensors. For example, a high-resolution camera may be replaced by a lower resolution camera, and the only change that may be required for the balance of the system is the degree of downsampling of the incoming video that is done.

As hinted at above, in the general the state of mind of the user may be reflected in various ways by the ‘state of mind’ of the intelligent agent of the system. For example, the ‘mood detector’ discussed above may be used as one of many inputs to the contextual center, which takes this into account by modifying its responses appropriately—by mirroring the user's rate of speech, expressing apology when the user is annoyed, expressing condolence if the users appear disconsolate, etc.

One example that illustrates capabilities of the system beyond those of current intelligent assistants is in providing oversight. For instance, a user may use the inventive system to oversee a car maintenance procedure. The avatar in this case has the goal or agenda of (for instance) assisting the user to change the car oil. The avatar of the system may give a series of steps to accomplish an oil change, starting with making sure the user has necessary equipment: 2 liters of AWE-50 oil (as determined by interrogating the user about his/her car, or consulting history data for that particular user), a 10 mm spanner wrench, a tub or pan to hold the used oil, and possibly a car jack or ramp. If this particular avatar has access to a camera, the avatar can verify that the spanner is the right size and the oil is suitable by having the user hold up these items to the camera, and using image processing subsystems to identify the spanner size and oil type. The avatar would then instruct the user to slide under the car with the spanner, the tub or pan, and a flashlight (or if the avatar is running on a smartphone, the smartphone flashlight would serve). Using the camera, the user would slide under the car under the avatar's instruction (e.g. by means of verbal instruction, ‘go left’, ‘continue forward’, etc.) as long as the avatar has enough information from the camera, and possibly context information about the car, to determine the location of the oil sump relative to the user. Once the user is underneath the oil sump (as determined by the avatar by means of imaging information from the camera), the avatar instructs the user to put the pan under the sump and unscrew the drainage bolt by turning it anti-clockwise with the spanner.

Such supervised tasks can take many forms, for instance supervising filling out forms (online or otherwise), learning skills like dancing or playing an instrument, cooking, solving homework or other problems, making sure a person takes a certain dose of medicine (instructions for which might be ‘1. shake the medicine bottle, 2. take a pill from the bottle and swallow it, 3. sit on a chair’) or does a certain exercise, and so on. The key point is that the domain knowledge and role of the avatar can be easily switched-on demand, with the inputs to the system (e.g. audio and video) allowing for the avatar to verify that given actions have occurred.

It is within provision of the invention to seamlessly transition between different sensors and communication platforms as the need arises.

It is within provision of the invention to initiate action (for instance a sales solicitation, task initiation, or the like) based on any possible input. For instance, if an agent of the invention that is adapted for medical diagnosis determines that a user has likely suffered a stroke, the system may immediately call for medical assistance, without any further action from the user. As another example, if an agent of the invention that is adapted for the role of a private tutor detects that the user hasn't understood a key concept even after several attempts, the agent may suggest taking a break e.g. to play a video game, before returning to the missed concept at a later stage.

It is within provision of the invention to detect and respond appropriately to ‘unexpected’ changes of context, such as when strangers edge into a conversation between agent and user, or the loss of the user's attention, change in focus of the user's conversation, etc.

It is within provision of the invention to improve the performance of existing chatbots and other agents. This can be done relatively easily, given the level of abstraction used by the system. For example, consider an existing agent having a set of relevant business information used by a textual chatbot, e.g., in the form of a script. This script can be copied and integrated into the conversation manager of the invention, and used directly.

It is within provision of the invention to respond to events in the scene, including verbal input from a user but also including any other input detected by the system. Changes in the scene can be integrated into the responses of the system. For instance, if an agent of the system is configured to supervise a student learning a vocabulary lesson requiring the student to write the translation of an English word into French, the agent may move on to the next word if a scene processing module taking input from the camera detects that the student has written the correct answer. As another example, the agent may recite a test question to which the student is expected to answer verbally. These examples illustrate a particularly felicitous use of the agent of the system, as an ‘infinitely patient’ tutor or teacher than can in principle be an ideal teacher for a number of subjects that require or benefit from ‘human’ interaction and attention, such as learning language (in which speaking to a fluent speaker (in this case an agent of the invention) capable of correcting mistakes is an excellent pedagogical model).

It is within provision of the invention to initiate interactions with users, for example by starting conversations with a person or people in the vicinity, writing text to a chat box, calling cellphones or landlines, sending emails, and so on. During these interactions it is further within provision of the invention to follow a proactive agenda, in order to steer the customer to a particular business outcome, like a sale.

It is within provision of the invention to use a variety of inputs including audio, video, chat, and combinations of these.

It is within provision of the invention to adapt to different languages by means of modular system components. For example, the language being used can be represented by various methods including use of a localized dictionary or language-specific NLP module.

It is likewise within provision of the invention to adapt to different businesses and sales models using similarly modular system components, including modules representing business information.

The avatar of the invention may be found to be particularly useful in therapeutic settings chiefly involving verbal interaction between a therapist or counselor and a patient. The avatar is in a sense the ‘perfect listener’ as it has infinite patience, perfect memory, and all the time in the world. Thus, it is within the provision of the invention to carry out or assist in the performance of various forms of counseling and therapy, including psychoanalysis, psychodynamics, behavioral therapy, cognitive therapy, humanistic therapy, and integrative therapy, or any combination for these. Counseling may be facilitated with the invention, including event counseling, performance counseling, professional growth counseling, and any other form of counseling.

It is within provision of the invention to perform a variety of therapies including behavior modification. To this end, the appearance and behavior of the avatar of the invention are carefully tailored in order to elicit a bond (possible including affection, empathy, friendship, desire for acceptance and/or approval, and the like) between the avatar and the user. Once the user is invested in the companionship of the avatar, various therapies can be performed more effectively. The appearance and behavior mentioned include a host of physical cues including eye contact, gestures and other body language, tone of voice, vocabulary chosen, cadence, facial expressions, and so on as may be gleaned from research in the psychology of human interaction, as well as testing, possibly using the system itself. It is further within provision of the invention to read the tone, vocabulary, speech, cadence, body language, physical gestures, facial expressions, tics and other bodily expressions of emotion of the user, in an attempt to deduce the mindset, emotional state, and other aspects of the user.

The testing mentioned above may include studying the effectiveness of a given element (of avatar appearance or behavior) on a number of subjects, or may be tested ‘on the fly’ on a given user to determine what gestures are effective and what are not, allowing for the system to learn a set of behaviors that are useful on a subject-by-subject basis.

Compliance, adherence, and capacitance are terms used to describe the degree to which a patient follows medical advice such as taking medication, but these terms can also apply to a host of other situations such as medical device use, self-care, self-directed exercises, therapy sessions, and adherence to various regimens such as those used to reduce or eliminate smoking or other addictions or habits, lose or maintain weight, fitness regimens, self-help programs, and the like. Diabetes and pre-diabetes management, maintenance of cognitive function, reducing procrastination, goal attainment, personal coaching, and so on. The common denominator of such programs is that an interaction takes place between a user and a caregiver (the coach, therapist, or the like), and this caregiver is replaced (or possibly assisted) by the avatar of the invention.

The behavior modification and cognitive behavioral therapy techniques of the invention may be based upon classical respondent and operant conditioning to change behavior, including positive and negative reinforcement adapted to promote or reinforce a given desired behavior, and/or administering positive and negative punishment and/or extinction with the goal of eliminating or reducing undesired behaviors.

It is within provision of the invention to use applied behavior analysis (ABA), based on the notion that cognition and emotions are covert behaviors that can be subjected to the same conditions as overt behavior. To this end, it is within provision of the invention to read body language, gestures, facial expressions, tics, and other bodily expressions of emotion in an attempt to deduce the covert behavior or state of the user.

It is within provision of the invention to use ‘flooding’ or desensitization to combat phobias, by means of exposing the user to the situations (objects, people, interactions or the like) that trigger the phobia, in a controlled and safe environment.

Other techniques and paradigms of behavior modification or therapy may also be employed, including for instance the ‘tell play do’ technique useful for children, live modelling. sensitization, desensitization, counterconditioning, and so on.

Since the invention may serve as a ‘pocket therapist’ there are some advantages it may have over a conventional counselor or therapist. In particular, any time a user is upset, frightened, tempted to engage in addictive behavior (including digital addictions), in need of support, looking for motivation, and so on, the avatar may be consulted at will to provide comfort, advice, motivation, or other forms of guidance.

Desensitization attempts to diminish emotional responses to a stimulus by repeated exposure to it, or when an emotional response is elicited in situations in which the emotion proves irrelevant or unnecessary. This process is useful for ‘unlearning’ phobias and reducing anxieties. A related method uses an ordered list of anxiety-evoking stimuli which can be used for gradual adaption, useful in particular for those experiencing depression or schizophrenia.

Since the avatar of the invention will generally be presented on a screen, the use of physical objects for purposes of desensitization is restricted but still possible. For example, an object that produces some undesirable reaction such as triggering a phobia, may be presented in a context tending to counter the negative associations the user may have with this object. Alternatively, the object or other objects may be used by the patient at the request of the avatar during sessions or thereafter, to reduce or overcome the phobia. Similarly, an object associated with addictive behavior, such as a cigarette, may be presented by the avatar in a negative context in an attempt to reduce the positive associations the user may have concerning cigarette smoking, for example by showing video of lung cancer patients who are so terribly addicted that they continue smoking through the breathing stoma in their necks after having undergone tracheotomy. Beyond the use of imagery on the part of the avatar, and the use or manipulation physical objects by the subject, the system producing the avatar image may also provide rewards in the form of visual imagery or the like. For example, when performing therapy with kids, the avatar may offer a child to play a game on the device being used to present the avatar. This game may serve in effect as a reward for following instructions, reducing unwanted behaviors, increasing desired behaviors, and so on.

Since the avatar of the invention may have access to detailed medical histories of one or more users as well as medical recommendations for different situations, it is within provision of the avatar of the invention that the avatar choose methodologies including verbal communications, prescription of particular regimes, and the like that are selected for likely effectiveness, based on the user's own medical history , demography, cohort, and so on. The multiple regimes may be combined or flavored based on the abovementioned history.

The avatar may also be used to document a user's history. The interactions a particular user has with an avatar of the invention may be stored for perusal later, either by the user him- or herself, by a human therapist or counselor, by medical professionals, and possibly by others, pursuant of course to privacy laws and similar restrictions that may be in place for such cases.

It is within provision of the invention that the avatar of the invention be used as a form of digital companion, for those with difficulty forming friendships, older people with fewer social interactions, and the like.

It is within provision of the invention that the avatar of the invention be used as a kind of digital reminder or secretary, as in principle it may have access (for example) to a set of personal data of the user such as emails, voice data e.g., from calls and digital meetings, and other personal data. Thus, a user may for instance be reminded of meetings, birthdays, or other time-sensitive occurrences, items to buy on a shopping trip, names of potential connections, and the like.

It is within provision of the invention that the avatar of the invention be used as a digital research assistant, gathering information on a subject described by the user and reporting summaries verbally and/or in the form of written text or graphics.

It is further within provision of the invention to use habituation techniques, by which the response to a stimulus decreases after repeated or prolonged presentations of that stimulus; immersion therapy adapted to allow a patient to overcome fears and phobias, anxiety, and panic disorders; and other such therapies.

For such purposes it is within provision of the invention to determine hierarchies, such as hierarchies of desires or fears that the user holds. To this end the avatar may ask the user a series of questions to determine (for example) the level of satisfaction that the object of an addiction gives in various situations, or the level of discomfort a fear causes in various conditions. These conditions can include, for instance, talking about the object of the addiction or fear, seeing a picture of it or watching a movie involving it, being in the same room with the object of their addiction or fear, physical contact with it, and so on. The therapy in this case involves moving the patient moves up the ‘hierarchy of fear’ by gradual desensitization, such as presenting a picture or movie of the object of fear, until the patient is able to cope with the fear directly.

It is within provision of the invention to perform or assist in the performance of various forms of behavior modification, including cognitive behavioral therapy, motivational interviewing, and any other treatments intended to improve adherence, compliance, and other aspects of behavior of a subject. It is within provision of the invention to use systematic desensitization, graduated exposure therapy, radical behaviorism, counterconditioning, relaxation and coping techniques, and similar therapies.

As mentioned, although there are physical limitations inherent in the use of an avatar, it can present visual and auditory stimuli and respond to such ‘seen’ and ‘heard’ through the sensors of the system, and thus in these respects may perform roughly equivalently to an online therapist. Beyond this the avatar may have certain advantages stemming from the fact that the avatar is always available, never forgets, and has no time or payment requirements.

In accordance with the aforementioned, it is within provision of the invention to assist in adhering to fitness regimens, diets and other forms of dietary behaviors, weight loss programs, smoking reduction or elimination plans, phobia elimination, substance abuse treatment, addiction treatment, behavioral support, and any other behavior modification technique traditionally involving person-person or person-to-group interaction. These assistance programs can be used as standalone programs, or in addition to other health, wellness or fitness plans, within sessions or between them.

Within this provision is also the orchestration of such combinations, including matching the agent's personality, look and feel with the user's or program's goals; deciding on schedule, including limiting or expanding the duration of each session, the time between sessions, or the time of day the sessions should be held at; suggesting new goals or skins to support current goals or other user's needs; deciding to drop goals or subgoals, split them to different agents, or sort them in order, and so on.

The foregoing description and illustrations of the embodiments of the invention has been presented for the purposes of illustration. It is not intended to be exhaustive or to limit the invention to the above description in any form.

Any term that has been defined above and used in the claims, should be interpreted according to this definition.

The reference numbers in the claims are not a part of the claims, but rather used for facilitating the reading thereof. These reference numbers should not be interpreted as limiting the claims in any form.

What is claimed is:

1. A method for conducting automated therapy comprising:
generating an artificial virtual conversation avatar capable of verbally and visually communicating with a user to provide at least behavioral, solution;
programming the avatar to direct a conversation with the user based on a predefined protocol;
conducting further conversations with the user over multiple sessions, such that a relationship is formed between the avatar and the user;
tracking, by the avatar, of the user's behavior and psychological measures over time using at least one behavioral model; and
guiding, by the avatar, of the user to comply with the therapy, based on the tracking of the user's behavior over time.

2. The method of claim 1, further comprising:
initiating at least one of contact with the user at useful times by the avatar; and
allowing the user to contact the avatar at will.

3. The method of claim 1, wherein the predefined protocol further comprises:
generating, by the avatar, an instinctual or contextual response based on a stimulus input from the user.

4. The method of claim 1, wherein tracking over time further comprises:
analyzing past conversations with the user; and
personalizing the interaction between the avatar and the user.

5. The method of claim 1, wherein the at least one behavioral model is any one of: cognitive-behavioral therapy, dialect-behavioral therapy, behavior modification therapy, motivational interviewing, interactional therapies, behavioral modification therapy, behavioral support, psychotherapy, 12-steps program, and any other behavior modification technique traditionally involving person-person or person-to-group interaction.

6. The method of claim 1, wherein the guiding, by the avatar, of the user to comply with the therapy further comprises:
encouraging at least one of adherence and compliance with the therapy;
encouraging the formation of new habits or the breaking of old habits; and
facilitating the user's adherence to any psychological, medical, or behavioral treatment.

7. The method of claim 1, wherein the relationship is guided towards cooperative interaction with the user.

8. The method of claim 1, further comprising:
measuring effectiveness of the avatar with respect to at least one metric, wherein the at least one metric is any one of:
a degree to which the user seeks more interaction, the user's evaluation of the avatar, the user's evaluation of the interactions with the avatar, the user's performance, the user's long-term commitment to a given program, the user's degree of behavioral change, and artificial-intelligence-based measures of engagement, retention and behavioral change of the user based upon the user's at least one of measured and reported behavior.

9. The method of claim 8, wherein characteristics of the avatar are varied from conversation to conversation to improve the at least one metric.

10. The method of claim 1, further comprising:
creating a behavioral solution that can be conducted over several therapy sessions,
wherein the behavioral solution is communicated to the user by the avatar.

11. The method of claim 1, wherein the avatar is rendered by a user device executing a set of modular subsystems each having inputs and outputs.

12. The method of claim 11, wherein the set of modular subsystems include:
an emotion detection engine for detecting voice and visual input from the user, including the user's tone, pitch, body language, and facial expressions; and
a language processing engine comprising neural-network Natural Language Processing (NLP), wherein the language process engine processes text and user emotional state as input, and generates text intended to achieve specific interaction goals as output.

13. The method of claim 11, further comprising:
training a language model on a corpus of text dealing with behavioral issues, wherein the behavioral issues include at least one of: self-help, fighting addiction, attitude adjustment, formation of good habits, phobia elimination, and increasing adherence.

14. The method of claim 1, wherein the behavioral solution further comprises any one of: cognitive, psychological, or any combination of thereof.

15. A non-transitory computer-readable medium storing a set of instructions for conducting automated therapy, the set of instructions comprising:
one or more instructions that, when executed by one or more processors of a device, cause the device to:
generate an artificial virtual conversation avatar capable of at least one of verbally and visually communicating with a user to provide a behavioral solution;
program the avatar to direct a conversation with the user based on a predefined protocol;
conduct further conversations with the user over multiple sessions, such that a relationship is formed between the avatar and the user;
track, by the avatar, of the user's behavior over time using at least one behavioral model; and
guide, by the avatar, of the user to comply with the therapy, based on the tracking of the user's behavior over time.

16. A system for conducting automated therapy comprising:
one or more processors configured to:
generate an artificial virtual conversation avatar capable of verbally and visually communicating with a user to provide a behavioral solution;
program the avatar to direct a conversation with the user based on a predefined protocol;
conduct further conversations with the user over multiple sessions, such that a relationship is formed between the avatar and the user;
track, by the avatar, of the user's behavior over time using at least one behavioral model; and
guide, by the avatar, of the user to comply with the therapy, based on the tracking of the user's behavior over time.
